SEQUENCE LISTING (9 sequences)

SEQ ID NO: 1 - RNA, 34 nt, Artificial Sequence
  AXL aptamer
augaucaaucgccucaauucgacaggaggcucac

SEQ ID NO: 2 - RNA, 17 nt, Artificial Sequence
  AXL aptamer
guacauucuagauagcc

SEQ ID NO: 3 - RNA, 28 nt, Homo sapiens
agucagugcaucacagaacuuugucuuu

SEQ ID NO: 4 - RNA, 28 nt, Homo sapiens
aggugaaguucuguuauacacucaggcu

SEQ ID NO: 5 - RNA, 17 nt, Homo sapiens
ggcuaucuagaauguac

SEQ ID NO: 6 - RNA, 34 nt, Artificial Sequence
  Scrambled aptamer
ggcgcuagaaccuucuaagcgaauacauuaccgc

SEQ ID NO: 7 - RNA, 34 nt, Homo sapiens; identical to SEQ ID NO: 6

SEQ ID NO: 8 - RNA, 26 nt, Homo sapiens
caaacuguacaggccacugccuugcc

SEQ ID NO: 9 - RNA, 17 nt, Homo sapiens
ggcuaucuagaauguac

(19) United States
(12) Patent Application Publication
(10) Pub. No.: US 2022/0356475 A1
(43) Pub. Date: Nov. 10, 2022

(54) CHIMERIC COMPLEX AND THERAPEUTIC USES THEREOF

(71) Applicants: UNIVERSITA' DEGLI STUDI DI TORINO, Torino (IT); CONSIGLIO NAZIONALE DELLE RICERCHE, Roma (IT)

(72) Inventors: Daniela TAVERNA, Chieri (Torino) (IT); Lorena QUIRICO, Venaria Reale (Torino) (IT); Francesca ORSO, Torino (IT); Vittorio DE FRANCISCIS, Napoli (IT); Carla Lucia ESPOSITO, Napoli (IT); Silvia CATUOGNO, San Giorgio a Cremano (Napoli) (IT)

(21) Appl. No.: 17/640,647

(22) PCT Filed: Aug. 31, 2020
(86) PCT No.: PCT/IB2020/058098

§ 371 Date: Mar. 4, 2022

(30) Foreign Application Priority Data
Sep. 6, 2019 (IT) 102019000015806

(51) Int. Cl. C12N 15/115; A61K 9/00; A61P 35/00

(52) U.S. Cl. CPC C12N 15/115; A61K 9/0019; A61P 35/00; C12N 2310/141; C12N 2310/16

(57) ABSTRACT

A chimeric complex comprising a microRNA in combination with an aptamer for AXL receptor tyrosine kinase is provided. Use of the chimeric complex for targeted treatment of a tumor disease, in particular in a therapy affecting onset and/or progression of tumor metastasis is also provided.

The present invention falls within the field of therapeutic treatments of tumor diseases, particularly solid tumors, more particularly tumor diseases characterized by high metastatic activity.

Tumor diseases are typically characterized by progression through successive, increasingly severe stages. In an initial stage, normal cells, as a result of genetic modifications, begin to proliferate abnormally in a microenvironment consisting of stromal cells embedded in a remodeled extracellular matrix infiltrated by immune cells. Cancer cells that acquire the ability to invade adjacent tissues, intravasate, move through the vascular system, stop in the capillaries and extravasate into the surrounding tissue parenchyma give rise to distant metastases. Since metastatic spread is responsible for over 90% of cancer-related deaths, a great effort in the field of clinical and pharmacological research is aimed at identifying appropriate therapies which allow metastatic development to be stopped or at least slowed down.

In recent years, miRNAs, i.e. small non-coding RNAs acting as negative post-transcriptional regulators for their target genes, have been shown to be involved in tumor biology. In particular, in a vast majority of cases, the formation and progression of tumor disease was found to be associated with aberrant expression of certain miRNAs, and this finding is supported by growing emerging evidence. The first paper on the role of miRNAs in cancer appeared in 2002 and referred to the consequences of miR-15 and miR-16 deletion in chronic lymphocytic leukemia (CLL) (Calin, G A, et al., (2002) “Frequent deletions and down-regulation of micro-RNA genes miR15 and miR16 at 13q14 in chronic lymphocytic leukemia”; Proc Natl Acad Sci 99: 15524-15529). Over 32,500 studies have been published since then. Among the plurality of miRNAs identified so far, let-7 miRNAs, miR-29 family, miR-34 and miR-148b were shown to act as suppressors of “tumors or metastases”, whereas the miRNA 17-92 cluster, miR-21, miR-10b and miR-214 were shown to play a role in promoting tumor growth or spread, depending on the tumor context. In particular, recent studies have shown that miR-148b controls breast cancer progression by coordinating a large number of target molecules, including ITGA5 integrin, its downstream players ROCK1 and PIK3CA/p110α (Cimino, D, et al. (2013) “miR148b is a major coordinator of breast cancer progression in a relapse-associated microRNA signature by targeting ITGA5, ROCK1, PIK3CA, NRAS, and CSF1”, FASEB J 27: 1223-1235) and the cell adhesion molecule ALCAM (Penna, E, et al. (2013) “miR-214 coordinates melanoma progression by upregulating ALCAM through TFAP2 and miR-148b downmodulation”, Cancer Res 73: 4098-4111). Furthermore, miR-148b expression was shown to be negatively regulated by the pro-metastatic miR-214, thus suggesting that miR-148b acts antagonistically in controlling the spread of breast cancer and melanoma (Orso, F, et al. (2016) “miR-214 and miR-148b Targeting Inhibits Dissemination of Melanoma and Breast Cancer”; Cancer Res 76: 5151-5162.).

Due to the role of miRNAs in cancer progression, cancer therapies based on their use have been developed which, however, suffer from major limitations due to the need for efficient in vivo delivery systems.

Therefore, there is an urgent need for new therapeutic strategies which are suited for targeting the progression of tumor diseases, thus making it possible to prevent, slow down and/or inhibit the onset of tumor metastases, while hampering the onset of any adverse side effects.

To meet these and other needs, the present invention provides the chimeric complex and pharmaceutical composition as defined in the appended independent claims.

Additional features and advantages of the invention are defined in the dependent claims, which form an integral part of the specification.

As will be apparent from the following detailed description, the present invention provides a chimeric complex which is defined by a combination of characteristics capable of giving said complex an effective antitumor activity accompanied by significant selectivity of action.

In particular, in the context of the present disclosure, the term “chimeric complex” refers to a macromolecular complex comprising two molecules of a different kind, in particular an artificial aptamer molecule and an isolated, naturally occurring miRNA molecule, which are capable of performing different actions.

The chimeric complex according to the invention comprises an aptamer directed towards the AXL receptor tyrosine kinase.

In the present disclosure, the term aptamer indicates a single-stranded DNA or RNA oligonucleotide molecule capable of binding to a certain target molecule, for example a cell transmembrane protein, with high affinity and selectivity. The AXL receptor, the target of the aptamer molecule according to the invention, is known to be expressed on the surface of a large prevalence of different tumor cells, where it exerts oncogenic activity.

The aptamer of the chimeric complex according to the invention comprises, from the 5′ end to the 3′ end:

(i) the nucleotide sequence SEQ ID NO. 1, as described in WO2012049108;

(ii) a linker element consisting of an unsubstituted linear alkyl chain containing from 4 to 20 carbon atoms; and

(iii) the nucleotide sequence SEQ ID NO. 2.

Preferably, the unsubstituted linear alkyl chain of the linker element contains from 6 to 18 carbon atoms, more preferably 12 carbon atoms.

The manufacture of an aptamer according to the invention falls well within the skills of those of ordinary skill in the art.

The chimeric complex according to the invention is also characterized in that it comprises a microRNA (miRNA) comprising: a “guide” strand consisting of the nucleotide sequence SEQ ID NO. 3 and a “passenger” strand consisting from the 5′ end to the 3′ end of the nucleotide sequence SEQ ID NO. 4 and the nucleotide sequence SEQ ID NO. 5.

In the present specification, the terms “microRNA” or “miRNA” refer to short endogenous non-coding RNA molecules with a length generally ranging from 20 to 25 nucleotides.

Within the scope of the present specification, the term “guide strand” refers, in particular, to the miRNA strand that is incorporated into the effector cytoplasmic complex, designated as RISC (RNA-Induced Silencing Complex), which guides the specific binding of the miRNA to the target RNA messenger molecule, thus mediating its gene silencing action. Likewise, the term “passenger strand” refers to the miRNA strand that does not associate with the RISC complex within the cell and is degraded.

In the chimeric complex according to the invention, the nucleotide sequence SEQ ID NO. 3 comprises miR-148b-3p sequence which is listed in the miRBase database with access number MIMAT0000759, and the sequence SEQ ID NO. 4 comprises miR-148b-5p sequence which is listed in the miRBase database with access number MIMAT0004699.

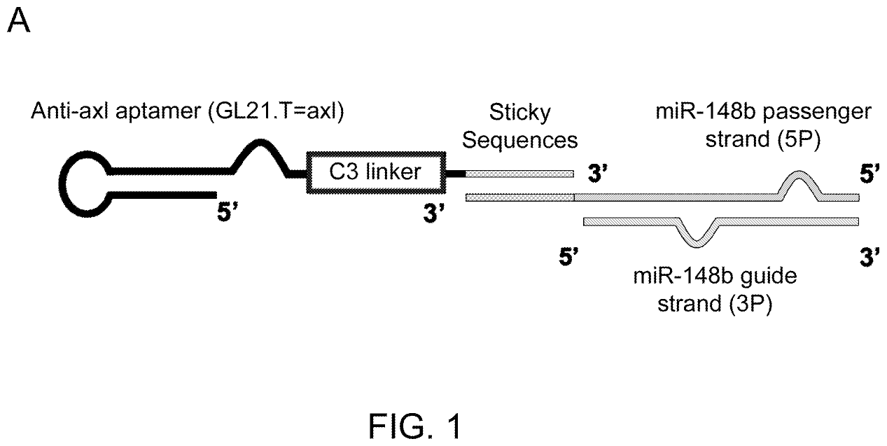

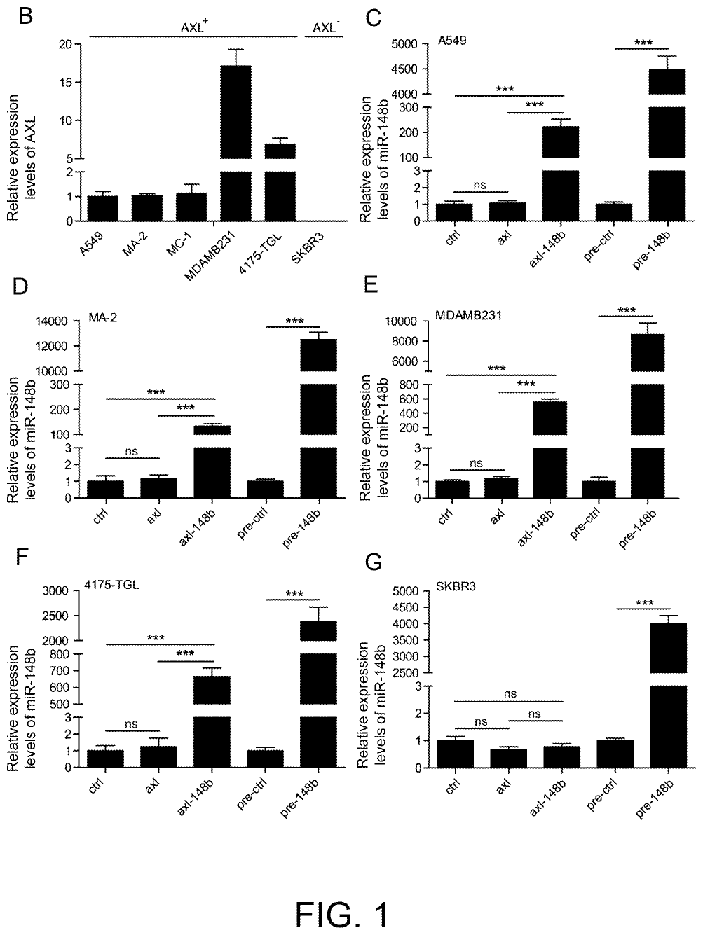

According to the invention, the nucleotide sequence SEQ ID NO. 2 at the 3′ end of the aptamer and the nucleotide sequence SEQ ID NO. 5 at the 3′ end of the “passenger” strand of the miRNA are complementary to each other. As a result, as shown in FIG. 1A, the annealing of these two nucleotide sequences allows the association of the aptamer with the miRNA, thereby the formation of the chimeric complex of the invention.

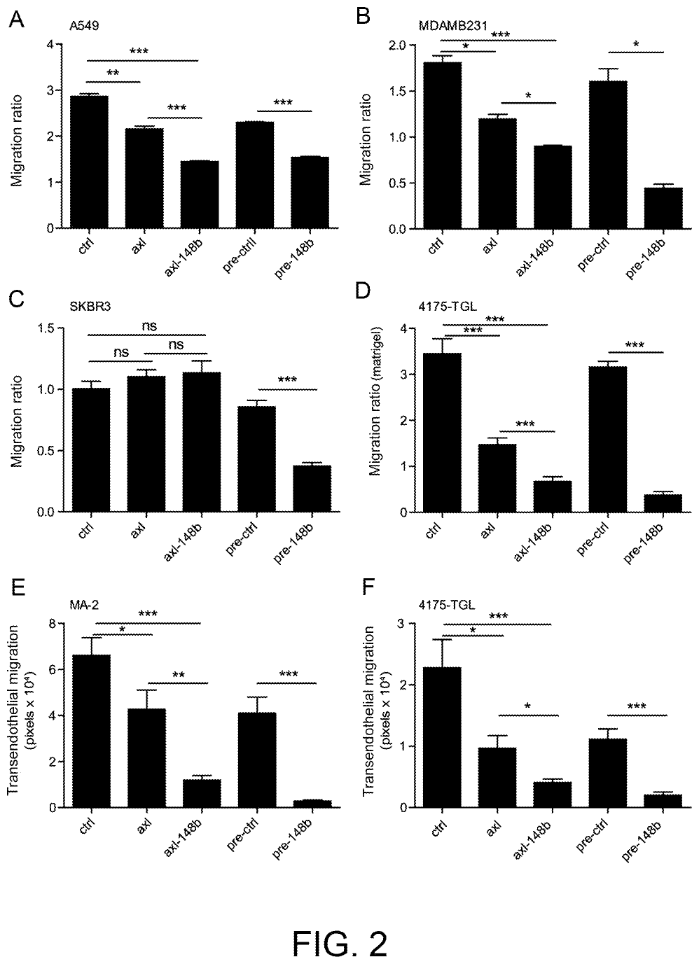

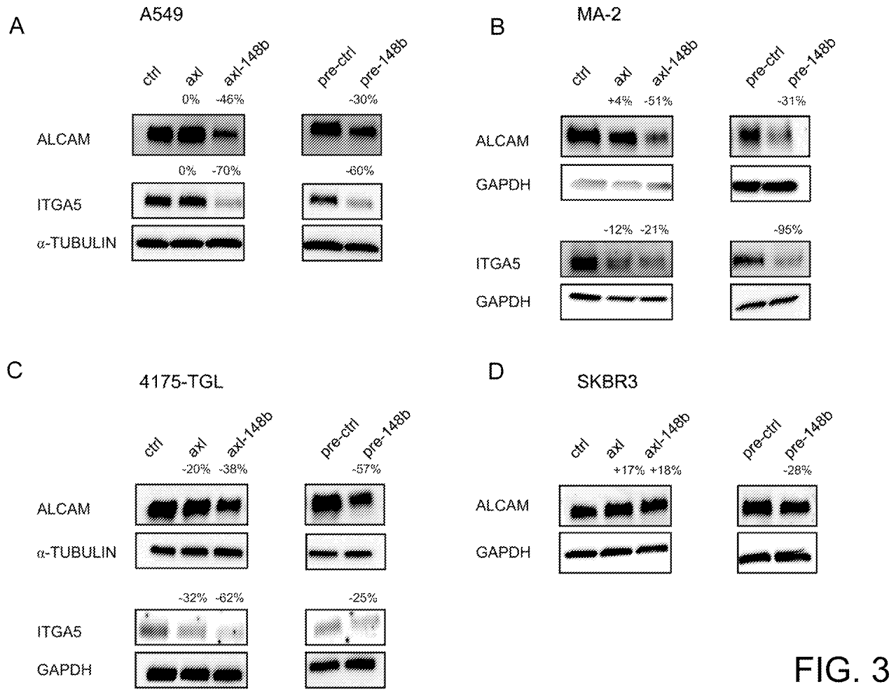

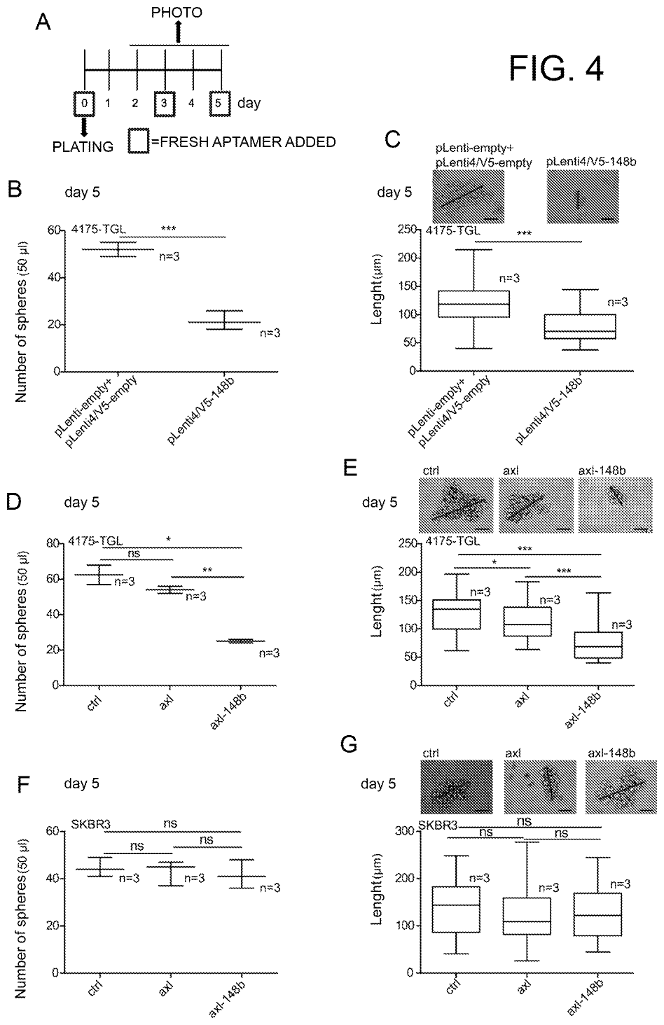

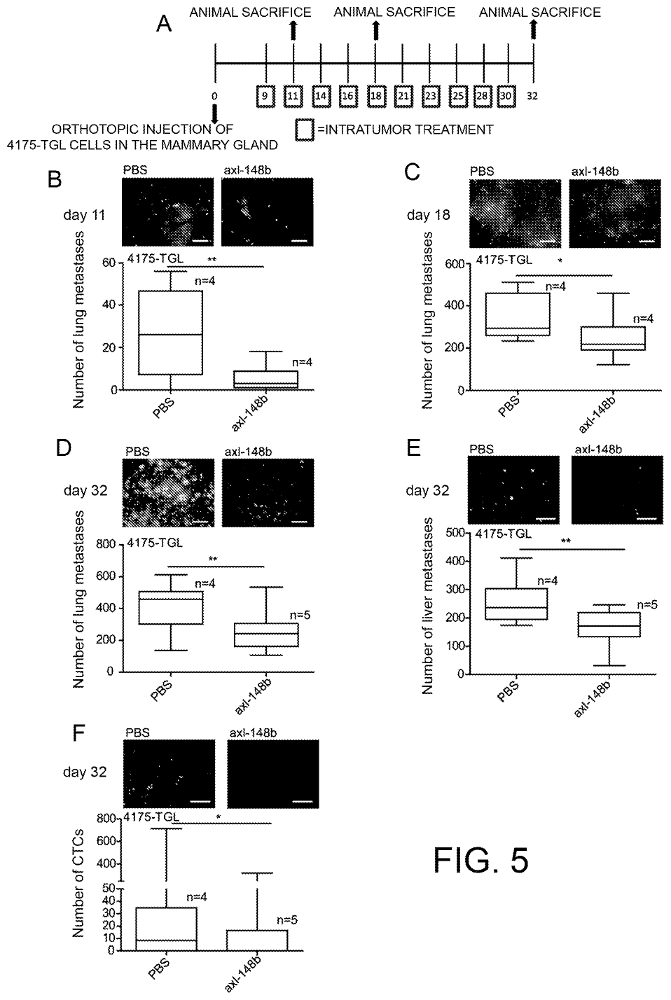

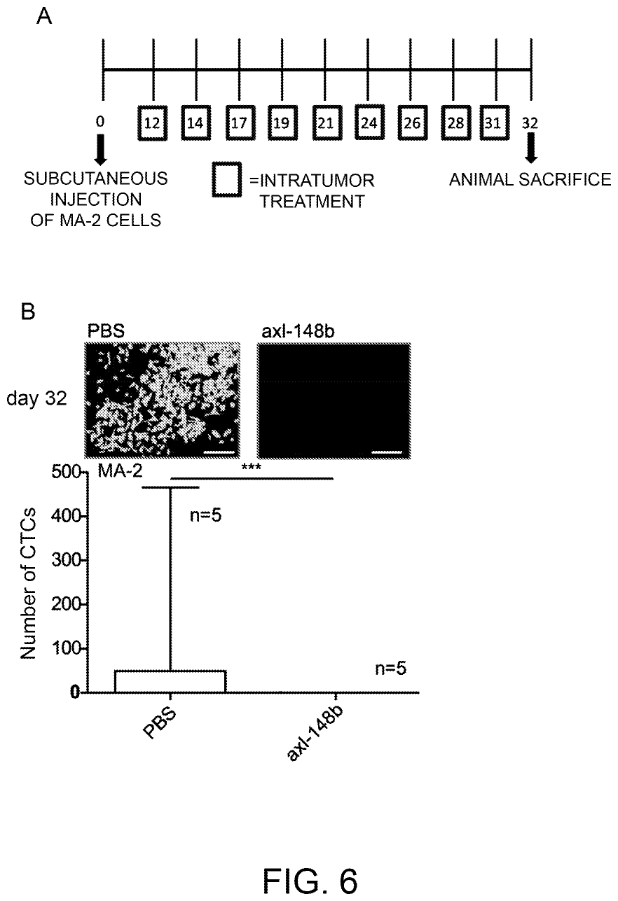

The present invention is based on the results obtained by the inventors in the experimentation and research activities described in the following experimental section. In short, in vitro studies carried out by the present inventors revealed that the use of the chimeric complex according to the invention leads to a significant decrease in the invasiveness and migratory capacity of tumor cells expressing the AXL receptor, while causing a significant decrease in the expression of miRNA target genes involved in tumor progression (FIGS. 2 and 3). Furthermore, treatment with the chimeric complex of the invention inhibits the formation and growth of breast cancer mammospheres in a three-dimensional in vitro model (FIG. 4). Importantly, the effects mentioned above only occur in cells that express AXL on their cell surface, not in cells that do not express this receptor. Even more importantly, the present inventors demonstrated that the chimeric complex according to the invention has high antitumor activity in vivo and, after direct administration in murine xenografts, is capable of inducing necrosis and apoptosis in primary tumor masses, as well as stopping the spread of tumor cells and the metastatic processes in these animals (FIGS. 5 and 6).

Studies carried out by the inventors have also highlighted the particular selectivity of action of the chimeric complex according to the invention as it is only active on tumor cells expressing the AXL receptor, not on AXL-negative neoplastic cells.

In the light of the above, the chimeric complex according to the invention represents an innovative therapeutic tool in the oncological field, which is particularly effective in counteracting tumor invasiveness and metastatic progression, and at the same time characterized by a considerable reduction of adverse side effects thanks to the particular selectivity of action of the aptamer which is capable of mediating the specific binding of said complex to the AXL-positive target tumor cell and its internalization.

Preferably, the chimeric complex according to the invention is nuclease-resistant.

More preferably, in the chimeric complex according to the invention, one or more pyrimidine base(s) of the nucleotide sequences SEQ. ID NO. 1, 2, 3, 4 and/or 5 is/are substituted with the corresponding 2′-fluoropyrimidine, and/or one or more purine base(s) of said nucleotide sequences is/are substituted with the corresponding 2′-O-methylpurine.

In one embodiment, in addition to the nucleotide substitutions previously described, or alternatively, in order to increase the nuclease-resistance, in the chimeric complex of the invention, the 3′ end of the nucleotide sequence SEQ ID NO. 2 and/or the 3′ end of the nucleotide sequence SEQ ID NO. 5 is/are locked by conjugation with a biotin molecule.

Further nucleotide sequence modifications suitable for providing the chimeric complex of the invention with nuclease-resistance include, for example, but are not limited to, the addition of 2′-amino (2′-NH2) ribose, monothiophosphates or thiophosphates, modifications to the phosphodiester bond (phosphorothioates and methylphosphonates), the use of phosphoramidates, 2′-O-alkyl ribonucleotides, replacement with locked nucleic acids (LNA) or peptide nucleic acids (PNA).

In another embodiment, the chimeric complex of the invention additionally comprises polyethylene glycol (PEG) or cholesterol in order to decrease renal clearance.

Thanks to its targeted anti-tumor, in particular anti-metastatic activity, the chimeric complex according to the invention is suitable for use in the therapeutic treatment of tumor diseases, preferably tumor diseases characterized by deregulated activity of the AXL receptor tyrosine kinase.

Tumor diseases include, for example, but are not limited to, melanoma, breast cancer and lung cancer.

A pharmaceutical composition comprising the chimeric complex of the invention as defined above, in combination with at least one pharmaceutically acceptable carrier, excipient and/or diluent, is also within the scope of the invention.

According to the invention, the pharmaceutical composition is suitable for use in the above therapeutic medical applications relating to the chimeric complex.

The pharmaceutical composition of the present invention can be formulated into any suitable dosage form, for example, for administration via the subcutaneous, intravenous, intraarterial, intraperitoneal, intramuscular, intranasal, or inhalation route.

In an alternative embodiment, the pharmaceutical composition according to the invention can be formulated into a dosage form suitable for local intratumoral administration, for example by injection under computed tomography guidance.

Of course, the selection of suitable carriers, excipients and/or diluents is carried out depending on the desired form of administration and this selection is within the skills of those of ordinary skill in the art. The selection of the active principle dose and dosage regimen also falls within the skills of those of ordinary skill in the art, and the selection thereof depends on several factors, such as for example the age and weight of the patient, the degree of progression of the disease, as well as the size of the tumor mass to be treated.

The invention is further described in the examples below, with reference to the accompanying drawings, wherein:

FIG. 1 shows the structure of the chimeric complex of the invention and its different ability to release miR-148b in AXL⁺ or AXL⁻ tumor cells. (A) Schematic representation of the chimeric complex. (B) qRT-PCR analysis of AXL mRNA levels in AXL⁺ cell lines: A549 lung adenocarcinoma cells, MA-2 and MC-1 melanoma cells, and MDAMB231 and 4175-TGL breast cancer cells, and in the AXL⁻ SKBR3 breast cancer cell line. The results are shown as changes in expression level (mean±SD) compared to A549 cells, normalized to GAPDH levels. (C-G) qRT-PCR analysis of miR-148b expression in the above cell lines, which were left untreated (controls=ctrl) or treated with 400 nmol/L of the aptamer (axl) or the chimeric complex of the invention (axl-148b). Alternatively, cells were transfected with 75 nmol/L of the miR-148b precursor (pre-148b) or its negative control (pre-ctrl). The results are shown as changes in expression level (mean±SD) compared to controls (ctrl or pre-ctrl), normalized to the levels of the U6 or U44 small nucleolar RNAs. One of two experiments performed in triplicate is shown for illustration. ns=non-significant; *p<0.05, **p<0.01, ***p<0.001. SD=standard deviation;

FIG. 2 shows that the chimeric complex of the invention is capable of inhibiting tumor cell motility. Migration assays in transwells were used for the experiments to assess the migration (A-C), invasive capacity through matrigel (D) or transendothelial migration through a monolayer of endothelial cells (HUVECs) plated on a porous membrane (E-F) of AXL⁺ or AXL⁻ tumor cells (as indicated in FIG. 1), which were left untreated (controls=ctrl) or treated with 400 nmol/L of the aptamer (axl) or the chimeric complex of the invention (axl-148b). Alternatively, cells were transfected with 75 nmol/L of the miR-148b precursor (pre-148b) or its negative control (pre-ctrl). The results are expressed as the ratio of the means±SEM of the area covered by the migrated/invasive tumor cells relative to the plated cells (A-D) or as the mean±SEM of the area (pixels) covered by the migrated cells (E-F). At least two independent experiments (in triplicate) were performed and representative results are shown. ns=non-significant; *p<0.05, **p<0.01, ***p<0.001. SEM=Standard Error Mean;

FIG. 3 shows that the chimeric complex of the invention modulates ALCAM and ITGA5 expression in cancer cells. (A-D) Western blot molecular analysis of the expression of the proteins ALCAM and ITGA5 in AXL⁺ (A-C) or AXL⁻ (D) tumor cells (as indicated in FIG. 1), which were left untreated (controls=ctrl) or treated with 400 nmol/L of the aptamer (axl) or the chimeric complex of the invention (axl-148b). Alternatively, cells were transfected with 75 nmol/L of the miR-148b precursor (pre-148b) or its negative control (pre-ctrl). Protein changes were calculated compared to controls (ctrl or pre-ctrl), normalized to loading controls (α-tubulin or GAPDH), and expressed as percentages (%). At least two independent experiments were performed, and representative results are shown;

FIG. 4 shows that the chimeric complex of the invention inhibits the formation and growth of mammospheres derived from AXL⁺ tumor cells but not from AXL⁻ tumor cells. (A) Experimental design followed in the mammosphere assays on 4175-TGL or SKBR3 breast cancer cell lines. The cells were plated, grown in suspension for 5 days and treated with 200/400 nmol/L of the aptamer or the chimeric complex of the invention on days 0, 3, 5, as indicated (numbers in squares). (B-C) Controls (pLenti-empty+pLenti4/V5-empty) or 4175-TGL breast cancer cells (pLenti4/V5-148b) over-expressing miR-148b were cultured (day 0, in suspension), and the formation and size of the spheres was assessed on day 5. (D-G) Formation and growth, at day 5, of spheres derived from 4175-TGL or SKBR3 breast cancer cells treated as described in (A). (B, D, F) Plots of the number of 4175-TGL or SKBR3 cell-derived mammospheres in 50 μl culture volume on day 5, reported as the mean±SEM. (C, E, G) Top: Representative images of day-5 derived mammospheres. Bottom: plots of the sizes of the spheres, reported as the mean±SEM of the length of the sphere (μm); the black lines correspond to the size measurements. (B-G) Two or three independent experiments (in triplicate) were performed, and representative results are shown. ns=non-significant; *p<0.05, **p<0.01, ***p<0.001. SEM=Standard Error Mean. Scale bar=25 μm (C, E, G);

FIG. 5 shows that the macromolecular complex of the invention prevents the spread of breast cancer in mice. (A) Experiment design: Red fluorescent (RFP-expressing) 4175-TGL cells were orthotopically injected into the mammary gland of NOD/SCID/IL2R mice, and the inventive chimeric complex or PBS was administered into the tumors starting from day 9 after cancer cell injection, when the masses were palpable (3 treatments/week, 300 pmoles in 100 μl, total of 10 injections, as indicated), and lung metastases were assessed at 11 (B), 18 (C) or 32 (D) days from cancer cell injections. Liver metastases (E) and circulating tumor cells (CTCs) (F) were also assessed on day 32. (B-F) Plots of the total number of lung and liver fluorescent metastases or of the CTCs, shown as the mean±SEM for the indicated number (n) of mice. Representative images of lung or liver fluorescent metastases or of the CTCs are shown. CTCs=Circulating Tumor Cells. *p<0.05, **p<0.01, ***p<0.001. SEM=Standard Error Mean. Scale bar=800 μm (B-E) or 50 μm (F);

FIG. 6 shows that the chimeric complex of the invention prevents melanoma spread in mice. (A) Experiment design: Red fluorescent (RFP-expressing) MA-2 cells were injected into the flank of NOD/SCID/IL2R mice, and the macromolecular complex of the invention or PBS was administered into the tumors starting from day 9 after injection, when the tumors were palpable (3 treatments/week, 300 pmoles in 100 μl, total of 9 injections, as indicated), and primary tumors or CTCs were analysed 32 days after cancer cell injections. (B) CTC assessment: Representative images are shown above the plots representing the total number of RFP positive cells obtained from blood (32 days post-injection), cultured for 7 days, represented as means±SEM for the indicated number (n) of mice. CTCs=Circulating Tumor Cells. Scale bar=50 μm (B). *p<0.05, **p<0.01, ***p<0.001. SEM=Standard Error Mean;

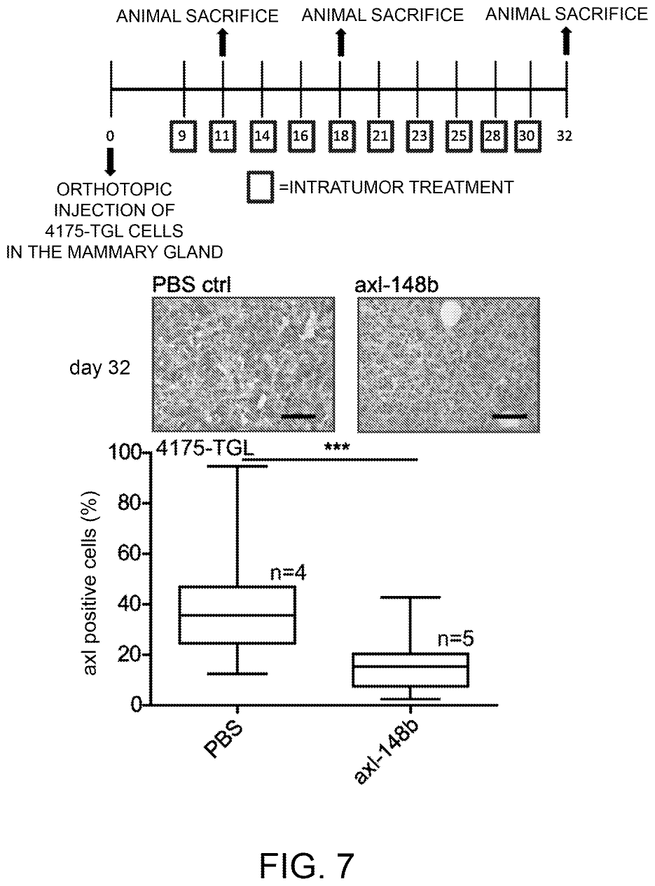

FIG. 7 shows the decrease in the number of AXL positive cells in primary murine tumors after treatment with the chimeric complex of the invention. Top: experiment design: red fluorescent (RFP-expressing) 4175-TGL cells were orthotopically injected into the mammary gland of NOD/SCID/IL2R mice, and the inventive chimeric complex or PBS was administered into the tumors starting from day 9 post-injection, when the masses were palpable (3 treatments/week, 300 pmoles in 100 μl, total of 11 injections, as indicated), and the level of AXL was determined 32 days after cancer cell injection. Bottom: (upper panel) representative photomicrographs of FFPE sections of primary tumors subjected to immunohistochemical staining with an anti-AXL antibody; (lower panel) plots representing the percentage (%) of positive cells over total cells, shown as the mean±SEM for the indicated number (n) of mice. 10 fields per mouse were assessed. IHC=immunohistochemistry. FFPE: formalin-fixed, paraffin-embedded. Scale bar=25 μm. *** p<0.001. SEM=Standard Error Mean;

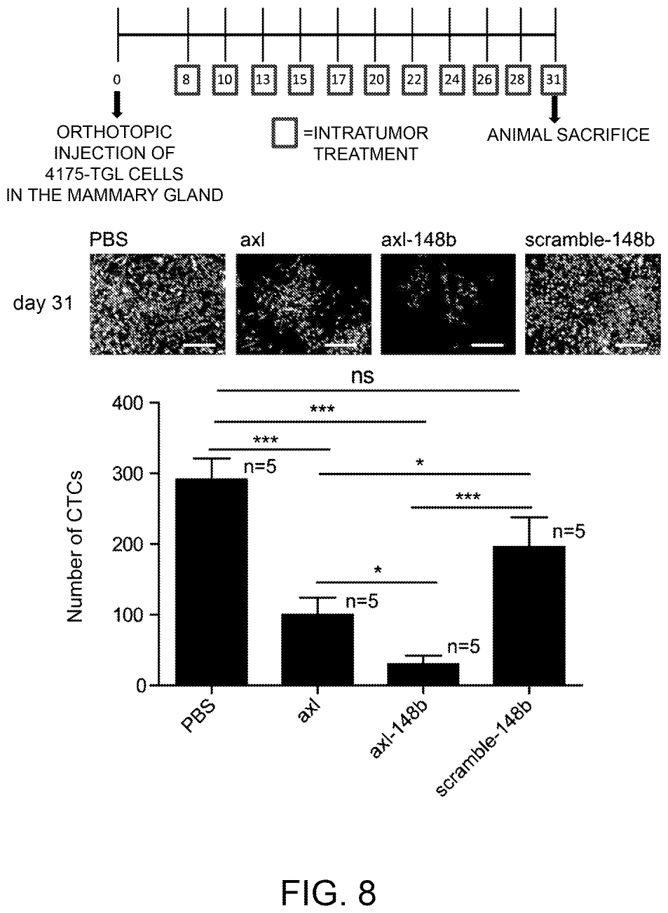

FIG. 8 shows the in vivo decrease in circulating tumor cells (CTC) after treatment with the macromolecular complex of the invention. Top: experiment design: red fluorescent (RFP-expressing) 4175-TGL cells were orthotopically injected into the mammary gland of NOD/SCID/IL2R mice, and either the chimeric complex of the invention (axl-148b), the scrambled complex (scramble-148b), the axl aptamer (axl), or PBS was administered into the tumors starting from day 8 post-injection, when the masses were palpable (3 treatments/week, 300 pmoles in 100 μl, total of 11 injections, as indicated), and the CTCs were measured 31 days after cancer cell injections. Bottom: (upper panel) representative photomicrographs of CTCs; (lower panel) plot representing the total number of CTCs as the mean±SEM for the indicated number (n) of mice. CTCs=Circulating Tumor Cells. Ns=non-significant *p<0.05, **p<0.01. SEM=Standard Error Mean. Scale bar=50 μm.

### EXAMPLES

Experimental Procedures

### Example 1: Cell Cultures

MA-2 melanoma cells (Xu, L, et al. (2008) “Gene expression changes in an animal melanoma model correlate with aggressiveness of human melanoma metastases”. Mol Cancer Res 6: 760-769.) were maintained as described in Penna, E, et al. (2011) “microRNA-214 contributes to melanoma tumour progression through suppression of TFAP2C”. EMBO J 30: 1990-2007; Penna, E, et al. (2013) “miR-214 coordinates melanoma progression by upregulating ALCAM through TFAP2 and miR-148b downmodulation”. Cancer Res 73: 4098-4111. MDAMB231, SKBR3 and A549 cells were purchased from the American Type Culture Collection (ATCC), while 4175-TGL cells were kindly provided by J. Massague (Minn, A J, et al. (2005) “Genes that mediate breast cancer metastasis to lung”. Nature 436: 518-524) and maintained under standard culture conditions. HUVEC cells (human endothelial cells obtained from the umbilical cord) were kindly provided by M. F. Brizzi and maintained as described in Penna, E, et al. (2011) “microRNA-214 contributes to melanoma tumour progression through suppression of TFAP2C”. EMBO J 30: 1990-2007; Penna, E, et al. (2013) “miR-214 coordinates melanoma progression by upregulating ALCAM through TFAP2 and miR-148b downmodulation”, Cancer Res 73: 4098-4111.

### Example 2: Reagents and Antibodies

The experiments described below used the following miR precursors: Pre-miR™ miRNA Precursor Negative Control No. 1, Pre-miR™ hsa-miR-148b miRNA Precursor (PM10264) (Applied Biosystems). The following reagents were used for the analysis of miRNA expression levels: MicroRNA TaqMan®: Hsa-miR-148b ID 000471, U6 snRNA ID001973, U44 snRNA ID001904 (Applied Biosystems). The following reagents were used for gene expression analysis: Quantitect Primer Assay: 218300Axl ID 33000 (Qiagen), Qiagen miScript-SYBR Green PCR Kit and miScript Primer Assay: hsa-let-7g ID 1 (Qiagen). The experiments used the following primary antibodies: anti-Cleaved Caspase-3 (Asp175) #9661 (Cell Signaling Technology), anti-Ki67 ab15580 (Abcam), anti-AXL (R&D Systems), anti-ITGA5 pAb RM10 (Molecular Biotechnology Center, University of Turin), anti-CD166/ALCAM mAb MOG/07 (Novocastra Laboratories), anti-GAPDH pAb V-18 (Santa Cruz Biotechnology), anti-α-tubulin mAb B5-1-2 (Sigma). The secondary antibodies used were as follows: HRP-conjugated goat anti-mouse IgG, goat anti-rabbit IgG (Santa Cruz Biotechnology), biotinylated goat anti-rabbit IgG, and biotinylated rabbit anti-goat IgG (Dako).

### Example 3: Transient Transfections and Production of Stable Cell Lines

In order to obtain miRNA transient expression and stable cell lines for the expression of the miRNAs, the present inventors followed the procedures described in Penna, E, et al. (2011) “microRNA-214 contributes to melanoma tumour progression through suppression of TFAP2C”. EMBO J 30: 1990-2007; Orso, F, et al. (2016) “miR-214 and miR-148b Targeting Inhibits Dissemination of Melanoma and Breast Cancer”. Cancer Res 76: 5151-5162.

### Example 4: Manufacture of the Chimeric Complex of the Invention and Controls

The manufacture of an aptamer according to the invention falls well within the skills of those of ordinary skill in the art.

In order to generate the chimeric complex of the invention, an miR-148b precursor was complexed with an AXL aptamer molecule. In short, the “guide” strand of miR-148b was annealed to the “passenger” strand. Then, the “passenger” strand of miR-148b and the AXL aptamer molecule were elongated at their 3′ ends with two 17-nucleotide sequences complementary to each other and annealed through their sticky ends.

The nucleotide sequences of the molecules used are shown below.

AXL Aptamer

(SEQ ID NO. 1)
5′AUGAUCAAUCGCCUCAAUUCGACAGGAGGCUCAC 3′;
(SEQ ID NO. 2)
5′ GUACAUUCUAGAUAGCC 3′;

miR-148b

“Guide” Strand (3P)

(SEQ ID NO. 3)
5′ AGUCAGUGCAUCACAGAACUUUGUCUUU 3′;

“Passenger” Strand (5P) (from the 5′ End to the 3′ End)

(SEQ ID NO. 4)
5′AGGUGAAGUUCUGUUAUACACUCAGGCU 3′;
and
(SEQ ID NO. 5)
5′GGCUAUCUAGAAUGUAC 3′.

During the experimental studies carried out by the present inventors, a scrambled aptamer and a chimeric axl-let-7g complex were used as controls.

In the context of the present description, the term “scrambled aptamer” is intended to refer to an aptamer molecule comprising a modified oligonucleotide sequence which, although capable of folding correctly, is however unable to bind and activate the AXL receptor tyrosine kinase.

The scrambled aptamer used as a control contains, from the 5′ end to the 3′ end, the following components:
the nucleotide sequence 5′GGCGCUAGAACCUUCUAAGCGAAUACAUUACCGC 3′ (SEQ ID NO. 6);
a linker element consisting of an unsubstituted linear alkyl chain containing 12 carbon atoms; and
the nucleotide sequence 5′ GUACAUUCUAGAUAGCC 3′ (SEQ ID NO. 2).

The chimeric axl-let-7g complex is described in Esposito C. L. et al, “Multifunctional Aptamer-miRNA Conjugates for Targeted Cancer Therapy”, (2014) Mol Ther. 22(6): 1151-1163.

In particular, this complex comprises the same aptamer as the chimeric complex of the invention, associated with the small let-7g RNA, the nucleotide sequences of which are shown below.

let-7g

“Guide” Strand

(SEQ ID NO. 7)
5′GGCUGAGGUAGUAGUUUGUACAGUUUG3′

“Passenger” Strand (from the 5′ End to the 3′ End)

(SEQ ID NO. 8)
5′CAAACUGUACAGGCCACUGCCUUGCC 3′;
and
(SEQ ID NO. 9)
5′GGCUAUCUAGAAUGUAC 3′.

In order to increase stability, in one embodiment of the macromolecular complex of the invention, one or more pyrimidine base(s) in the nucleotide sequences has/have been substituted with the corresponding 2′-fluoropyrimidine and/or one or more purine base(s) has/have been substituted with the corresponding 2′-O-methylpurine.

The RNA molecules described above were synthesized at the Synthetic and Biopolymer Chemistry Core, Beckman Research Institute, City of Hope, Duarte, Calif.

The “guide” strand of miR-148b contains two protruding bases (UU) at the 3′ end to facilitate the processing mediated by the Dicer enzyme.

The following experimental procedure was carried out in order to prepare the chimeric complex of the invention, the complex containing the scrambled aptamer, or the axl-let-7g complex: (i) the “passenger” and “guide” strands of miR-148b or let-7g were annealed after incubation in annealing buffer at 95° C. for 10 minutes, at 55° C. for 10 minutes and then at 37° C. for 20 minutes; (ii) the aptamers containing the sticky ends or the scrambled sequences were folded (5 minutes 85° C., 3 minutes on ice, 10 minutes at 37° C.); (iii) equal amounts of aptamer/scramble and paired “guide” and “passenger” strands were then annealed again by incubating them together at 37° C. for 30 minutes. Annealing efficiency was checked as described in Catuogno, S, et al. (2015) “Selective delivery of therapeutic single strand antimiRs by aptamer-based conjugates”. J Control Release 210: 147-159. In order to treat the cells with the chimeric complexes described above, the cells were plated in 24-well plates at 80% confluence and treated 24 hours later with the folded aptamers by adding them to their culture medium.

### Example 5: Protein or RNA Isolation, Western Blot, qRT-PCR

The procedures for obtaining total protein or RNA extracts, and the Western Blot (WB) and qRT-PCR assays were performed as described in Penna, E, et al. (2011) “microRNA-214 contributes to melanoma tumour progression through suppression of TFAP2C”. EMBO J 30: 1990-2007.

### Example 6: Proliferation, Migration, Invasion and Transendothelial Migration Assays

In vitro cell proliferation, migration, invasion and transendothelial migration assays were performed as described in Penna, E, et al. (2011) “microRNA-214 contributes to melanoma tumour progression through suppression of TFAP2C”. EMBO J 30: 1990-2007; Penna, E, et al. (2013) “miR-214 coordinates melanoma progression by upregulating ALCAM through TFAP2 and miR-148b downmodulation”. Cancer Res 73: 4098-4111; Cerchia, L, et al. (2012) “Targeting Axl with an high-affinity inhibitory aptamer”. Mol Ther 20: 2291-2303.

### Example 7: Mammosphere Formation Assays

Mammosphere formation assays were performed as described at https://www.stemcell.com/tumorsphere-culture-human-breast-cancer-cell-lines-lp.html, on 24-well plates coated with poly-HEMA (poly-2-hydroxyethyl methacrylate) using two different protocols. According to a first protocol, single breast cancer cells (8×10³ cells/well for the 4175-TGL cells, 1×10⁴ cells/well for the SKBR3 cells) were plated (day 0), maintained in suspension in MammoCult Medium (StemCell Technologies) and left untreated (controls=ctrl) or treated with 400 nmol/L of the axl aptamer or the chimeric complex of the invention. Treatments were repeated on days 3 and 5 (200 nmol/L). On day 5, the size and number of the spheres were assessed by using a Zeiss AxioObserver microscope (Zeiss) and the ImageJ software (http://rsbweb.nih.gov/ij/). For assessing the size, the long side of the spheres (length) was measured. For assessing the number, the total number of spheres was counted in 50 μl volume for each treatment.

According to an alternative protocol, single cells were plated and maintained as described above, and the mammospheres were dissociated on day 5, counted, plated again at the same density and treated in the same way. The spheres were analysed on day 12. In some experiments, cells were labelled on day 5 with PKH26 (Sigma, 10-7M, 5 min) and the percentage (%) of PKH26 positive cells was analysed on day 12 by FACS analysis after dissociation of the mammospheres to evaluate stemness. FACSCalibur was used to measure PKH26 positive cells over the total (100%).

### Example 8: Histology and Immunohistochemistry

5 μm thick tissue sections were cut from formalin-fixed, paraffin-embedded (FFPE) tumor specimens and stained with hematoxylin and eosin (H&E) for standard histological observations. Immunohistochemical staining (IHC) was performed by using anti-Ki67, anti-cleaved caspase 3 or anti-axl antibodies, with avidin-biotin-peroxidase techniques (Anti-Mouse HRP-DAB Cell & Tissue Staining Kit, R & D Systems). The slides were counterstained with hematoxylin.

### Example 9: Stability of the Chimeric Complex of the Invention in Human Serum

The stability of the chimeric complex of the invention in human serum was assessed as described in Catuogno, S, et al. (2015) “Selective delivery of therapeutic single strand antimiRs by aptamer-based conjugates”. J Control Release 210: 147-159.

### Example 10: In Vivo Tumor Growth and Metastasis Assays

All experiments performed with animals were performed in compliance with ethics. NOD/SCID/IL2R_null (NSG) mice were injected with tumor cells as described in Penna, E, et al. (2013) “miR-214 coordinates melanoma progression by upregulating ALCAM through TFAP2 and miR-148b downmodulation”. Cancer Res 73: 4098-4111; Orso, F, et al. (2016) “miR-214 and miR-148b Targeting Inhibits Dissemination of Melanoma and Breast Cancer”. Cancer Res 76: 5151-5162. The tumors, once palpable, were treated with PBS or with the chimeric complex of the invention (300 pmol/injection, three injections per week). Mice were sacrificed and analysed 11, 18 or 32 days after injections of MA-2 or 4175-TGL cells, respectively. The weight and morphology of the primary tumor and the lung or liver metastases were assessed as described in Penna, E, et al. (2013) “miR-214 coordinates melanoma progression by upregulating ALCAM through TFAP2 and miR-148b downmodulation”. Cancer Res 73: 4098-4111; Orso, F, et al. (2016) “miR-214 and miR-148b Targeting Inhibits Dissemination of Melanoma and Breast Cancer”. Cancer Res 76: 5151-5162. Organ size (liver, spleen, kidney) (weight) and morphology (hematoxylin and eosin staining) were analysed at the end point.

### Example 11: Isolation of Circulating Tumor Cells

Circulating tumor cells (CTCs) were isolated as described in Dettori, D, et al. (2018) “Therapeutic Silencing of miR-214 Inhibits Tumor Progression in Multiple Mouse Models”. Mol Ther. 26(8):2008-2018.

### Example 12: Statistical Analysis

All results are presented as the mean±Standard Deviation (SD) or ±Standard Error Mean (SEM), as indicated, and the two-tailed Student's t-test was used for comparisons. *=p<0.05; **=p<0.01; ***=p<0.001 were considered statistically significant. ns=indicates a non-statistically significant p-value.

Results

### Example 13: Axl Aptamer-Mediated miR-148b Transport by Using the Chimeric Complex of the Invention

The present inventors assessed, by electrophoretic analysis on non-denaturing gel, the efficiency of pairing of the complex of the invention with the complementary sequences at the 3′ ends of the aptamer molecule and the “passenger” strand of the miRNA, as well as the pairing of the “guide” strand with the “passenger” strand.

As demonstrated by qRT-PCR analysis and shown in FIG. 1, A549 lung adenocarcinoma cells, MA-2 and MC-1 melanoma cells, and MDAMB231 and 4175-TGL breast cancer cells express AXL (AXL+), whereas SKBR3 breast cancer cells do not (AXL−). Therefore, AXL+ or AXL− cells were treated with the chimeric complex of the invention, and miR-148b levels were measured by qRT-PCR analysis 48 hours after treatment and compared with those of untreated cells (controls=ctrl) or of cells treated with the aptamer molecule alone (axl).

Alternatively, the same cell types were transfected with pre-miR-148b (pre-148b) or pre-control (pre-ctrl).

Importantly, as shown in FIGS. 1C-G, after treatment with the chimeric complex of the invention, a significant increase in miR-148b expression was found in AXL+ cells, but not in AXL− cells, compared to controls and cells treated with the aptamer molecule alone, and the increase in expression appeared to be dose- and time-dependent. No increase in miR-148b levels was observed in AXL+ tumor cells when using a chimeric complex in which the aptamer molecule is complexed with another small RNA, let-7g.

In contrast, a significant increase in miR-148b levels was detected in all cells transfected with pre-148b, but not with pre-ctrl, including SKBR3 cells which are AXL−. In order to further verify the specificity of transport of miR-148b by AXL, the present inventors generated a chimeric scrambled complex in which an aptamer molecule with a scrambled sequence has been complexed with miR-148b. In this case, the scrambled sequence corresponds to a modified oligonucleotide sequence capable of folding correctly, but incapable of binding and activating the AXL receptor tyrosine kinase. When AXL+ cells were treated with scrambled aptamer molecules or scrambled chimeric complexes, no changes in miR-148b levels were observed, similar to the control samples.

The above results demonstrate the specific transport of miR-148b into AXL+ cells by using the chimeric complex of the invention.

### Example 14: The Chimeric Complex of the Invention Inhibits the Movement of Cancer Cells but does not Affect their Proliferative Capacity

In order to assess the effects of the chimeric complex of the invention on metastatic traits, A549 lung adenocarcinoma cells, MDAMB231, 4175-TGL and SKBR3 breast cancer cells, or MA-2 melanoma cells were left untreated (ctrl) or treated with the scrambled aptamer or with the axl aptamer or with the chimeric complex of the invention, or alternatively were transfected with miR-148b precursors (pre-148b) or controls (pre-ctrl).

Migration, invasion through Matrigel, transendothelial migration through a HUVEC monolayer and proliferation assays were performed on the treated cells.

As shown in FIG. 2A-F, an inhibitory effect on cell motility was observed for all AXL+ cells, but not for AXL− SKBR3 cells, treated with the axl aptamer alone or with the chimeric complex of the invention compared to control cells (ctrl or scrambled), and the inhibitory effect was greater when the chimeric complex of the invention was employed.

A similar effect was observed for cells transfected with pre-148b versus pre-ctrl, both for AXL+ and AXL− cells, indicating that the biological effects of miR-148b after administration of the chimeric complex of the invention are mediated by transport by the axl aptamer, and therefore specific for AXL-expressing cells.

Surprisingly, the present inventors did not detect any effect on cell proliferation when the cells were treated with the chimeric complex of the invention or with the axl aptamer, or when transfected with pre-148b compared to controls (ctrl or pre-ctrl), indicating that the effect of miR-148b is mainly performed on cell movement.

Since cells treated with scrambled or untreated (ctrl) molecules gave similar results in motility tests, the present inventors considered ctrl samples as negative controls in the experiments described below.

### Example 15: The Chimeric Complex of the Invention Affects the Direct Targets of mir-148b in Cancer Cells

With the aim of investigating the molecular mechanism underlying the effects of the chimeric complex of the invention on metastatic traits, the present inventors analysed the expression of ALCAM and ITGA5, which are two direct targets of miR-148b capable of coordinating extravasation of cancer cells.

A549 lung adenocarcinoma cells, MA-2 melanoma cells or 4175-TGL and SKBR3 breast cancer cells were treated with the chimeric complex of the invention or with the axl aptamer alone, and the expression of ALCAM and ITGA5 proteins compared to control cells (ctrl) or cells transfected with miR-148b precursors (pre-148b) or controls (pre-ctrl) was determined by Western Blot analysis.

As shown in FIG. 3A-D, the expression of ALCAM and ITGA5 is reduced in AXL⁺ cells, but not in AXL⁻ cells, treated with the chimeric complex of the invention. Instead, all cell types showed a reduction in the levels of these two adhesion molecules when transfected with pre-148b, but not with pre-ctrl.

In summary, the results obtained by the present inventors indicate that the chimeric complex of the invention acts on the coordination of molecular pathways involved in cell dissemination.

### Example 16: The Chimeric Complex of the Invention Affects the Number and Size of the Mammospheres

Since cancer stem cells (CSCs) are responsible for metastatic spread, the present inventors investigated the influence of the inventive chimeric complex on the development of 3D mammospheres derived from 4175-TGL and SKBR3 breast cancer cells. For this purpose, single cells were plated on day 0, left untreated (controls=ctrl), or treated on days 0, 3 and 5 with the chimeric complex of the invention or with the axl aptamer alone, and the mammospheres were analysed on day 5 (FIG. 4A).

According to an alternative procedure, single cells were plated on day 0 and the derived mammospheres were dissociated on day 5, plated again, and left untreated (controls=ctrl) or treated on days 5, 8 and 10 with the chimeric complex of the invention or with the axl aptamer alone; the mammospheres were then analysed on day 12.

In all experiments, qRT-PCR analysis showed that miR-148b levels were increased in AXL⁺4175-TGL breast cancer cells, but not in AXL⁻ SKBR3 cells, following treatment with the chimeric complex of the invention, compared to cells treated with the control or the axl aptamer. In parallel, 4175-TGL cells over-expressing miR-148b (pLenti4/V5-148b) and empty controls (pLentiempty+pLenti4/5V-empty) were also plated on day 0, and the mammospheres were analysed on day 5. As shown in FIG. 4B-C, the present inventors found that pLenti4/V5-148b-expressing cells generated a reduced number of spheres of a smaller size compared to the pLenti-empty+pLenti4/5V-empty controls. Similarly, when the number or size of the spheres or the percentage (%) of PKH26 positive cells in 4175 cell-derived mammospheres were determined on day 5 (FIG. 4B-E) or day 12, following the treatments described above, a strong reduction in stemness was observed in spheres treated with the chimeric complex of the invention compared to cells treated with the control or the axl aptamer. Instead, when the chimeric complex of the invention was used to treat mammospheres derived from AXL− SKBR3 cells, no effect on number or size of the mammospheres was detected (FIG. 4F-G). In summary, the data produced by the present inventors show that the chimeric complex of the invention affects the formation and growth of mammospheres in an AXL-dependent manner, similar to cells over-expressing miR-148b, indicating that the chimeric complex of the invention is effective in blocking cancer cell stemness and subsequent metastasization.

### Example 17: The Chimeric Complex of the Invention Blocks Cancer Cell Dissemination

In order to evaluate the efficacy of the chimeric complex of the invention on primary tumors and metastatic dissemination in mice, tRFP-positive 4175-TGL breast cancer cells or MA-2 melanoma cells were injected orthotopically into the mammary gland and the flank (subcutaneously), respectively, of NSG immunocompromised mice, and the mice were administered with the chimeric complex of the invention or PBS (control), 3 times a week, starting from when the tumors were palpable (day 9 for 4175-TGL, and 12 for MA-2), as shown in FIGS. 5 and 6A. The chimeric complex of the invention was administered directly into the xenografts and not intravenously. Primary tumor characteristics, metastasis formation, and circulating tumor cells (CTCs) were analysed at day 11, 18, or 32 post-injection. Hematoxylin and eosin staining showed an increase in tumor necrosis, particularly in AXL⁺ tumor cells (FIG. 7), while immunohistochemical (IHC) analysis for cleaved caspase-3 and Ki67 revealed increased apoptosis in neoplastic cells, but no change in proliferation. Even more importantly, a significant reduction in lung metastasis formation was observed in mice treated with the chimeric complex of the invention compared to controls at days 11, 18 and 32 for 4175-TGL (FIG. 5B-D). A marked reduction in liver metastases and CTCs was also detected at days 31 and 32 in these animals compared to control groups treated with the axl aptamer or with the scrambled chimeric complex (FIG. 5E and FIG. 8). It is also noteworthy that the chimeric complex (axl-148b) is more capable of blocking CTCs than the axl aptamer alone (FIG. 8).

In parallel, a decrease in CTCs was observed at day 32 in mice bearing MA-2-derived primary tumors treated with the chimeric complex of the invention (FIG. 6B). Relevantly, following the administration of the chimeric complex of the invention, no toxicity was detected in the animals compared to controls. In fact, no morphology alterations (analysed by hematoxylin and eosin staining) or weight alterations in the liver, spleen and kidneys were detected at the end of the experiment, as in FIG. 5A. Together, the above results demonstrate that the chimeric complex of the invention is a powerful therapeutic tool capable of affecting primary tumor dissemination, with no signs of toxicity and high clinical potential.

1. An isolated chimeric complex comprising:
a) an aptamer directed towards an AXL receptor tyrosine kinase, said aptamer comprising from the 5′ end to the 3′ end:
(i) the nucleotide sequence of SEQ ID NO. 1;
(ii) a linker element, said linker element consisting of an unsubstituted linear alkyl chain containing from 4 to 20 carbon atoms; and
(iii) the nucleotide sequence of SEQ ID NO. 2; and
b) a microRNA (miRNA) comprising a “guide” strand consisting of the nucleotide sequence of SEQ ID NO. 3 and a “passenger” strand consisting from the 5′ end to the 3′ end of the nucleotide sequence of SEQ ID NO. 4 and the nucleotide sequence of SEQ ID NO. 5;
wherein the nucleotide sequences of SEQ ID NO. 2 and SEQ ID NO. 5 are complementary to each other.

2. The isolated chimeric complex of claim 1, wherein the unsubstituted linear alkyl chain contains 12 carbon atoms.

3. The isolated chimeric complex of claim 1, wherein the isolated chimeric complex is nuclease-resistant.

4. The isolated chimeric complex of claim 3, wherein one or more pyrimidine bases in the nucleotide sequences of SEQ ID NO. 1, 2, 3, 4 and/or 5 are substituted with a corresponding 2′-fluoropyrimidine.

5. The isolated chimeric complex of claim 3, wherein one or more purine bases in the nucleotide sequences of SEQ ID NO. 1, 2, 3, 4 and/or 5 are substituted with a corresponding 2′-O-methylpurine.

6. A method for the treatment of a tumor disease in a subject in need thereof, said method comprising administering to said subject, the isolated chimeric complex claim 1.

7. The method of claim 6, wherein the tumor disease is characterized by deregulated activity of an AXL receptor tyrosine kinase.

8. The method of claim 6, wherein the tumor disease is selected from the group consisting of melanoma, breast cancer and lung cancer.

9. The method of claim 6, wherein the treatment is a therapy affecting onset and/or progression of metastasis.

10. A pharmaceutical composition comprising an isolated chimeric complex according to claim 1, and at least one pharmaceutically acceptable vehicle, excipient and/or diluent.

11. The pharmaceutical composition of claim 10, wherein the pharmaceutical composition is in a pharmaceutical form suitable for intratumor administration.

12. The pharmaceutical composition of claim 10, wherein the pharmaceutical composition is in a pharmaceutical form suitable for administration via the subcutaneous, intravenous, intraarterial, intraperitoneal, intramuscular, intranasal, or inhalation route.
